(19) United States
(12) Patent Application Publication
(10) Pub. No.: US 2020/0082324 A1
(43) Pub. Date: Mar. 12, 2020

(54) System, Method, and Computer Program for Virtual Inventory in Optometric Doctor Practices

(71) Applicant: EyeOn.pro Ltd, Timnath, CO (US)

(72) Inventors: Craig Dean Hanson, Elk Grove, CA (US); Paul Aaron DeVoe, Timnath, CO (US)

(21) Appl. No.: 16/126,715

(22) Filed: Sep. 10, 2018

(51) Int. Cl. G06Q 10/08; G16H 10/60

(52) U.S. Cl. CPC G06Q 10/087; G16H 10/60

(57) ABSTRACT

The presently disclosed technology includes a system, method, and computer program product provided for changing the timing of selection of optometric frame inventory sufficient enough to enable virtual inventory that can be fulfilled in time to match the traditional optometric experience of trying on glasses frames in the optometric practice.

### BACKGROUND

Eyewear sales typically are an important revenue stream to the optometric practice. While at an optometric practice appointment, the practice of frame selection and purchasing by a patient is physically browsing through frame inventory. At that time, the patient may review the available physical inventory, select, and try on frames. In this environment, the consumer is limited to an inventory selection of the physical inventory that an optometric practice has purchased and has on hand.

As the optometric practice moves their physical inventory to digital selection there is an opportunity to present hundreds of frame options without requiring physical retail space to display or capital expenditures to purchase the frames. The process enables significant data capture and uses deep trending information, comparison information, and statistics.

### FIELD OF THE INVENTION

The present invention relates generally to inventory in an optometric practice.

### SUMMARY

A system, method, and computer program product are provided to offer digital inventory to an optometry practice's patients. The virtual inventory selections can be physically delivered to the practice before the patient visit. This model enhances the patient's selection and maintains the shopping value of physically trying on frames in the optometry office.

### BRIEF DESCRIPTION OF THE DRAWINGS

FIG. 1 illustrates an exemplary system, in accordance with one embodiment.

FIG. 2 illustrates a network architecture, in accordance with one possible embodiment

FIG. 3 illustrates a method for offering patient access to virtual inventory selections that can be physically delivered to the practice before the patient visit.

### DETAILED DESCRIPTION

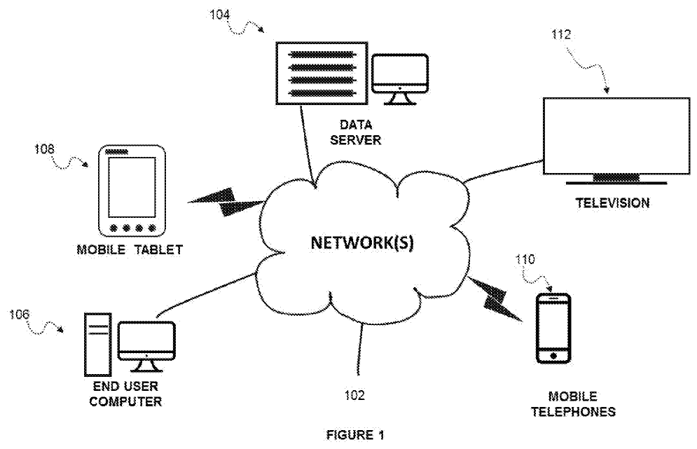

FIG. 1 illustrates a network architecture 100, in accordance with one possible embodiment. As shown, at least one network 102 is provided. In the context of the present network architecture 100, the network 102 may take any form including, but not limited to a telecommunications network, a local area network (LAN), a wireless network, a wide area network (WAN) such as the Internet, peer-to-peer network, cable network, etc. While only one network is shown, two or more similar or different networks 102 may be provided.

Coupled to the network 102 is a plurality of devices. For example, a server computer 104 and an end user computer 106 may be coupled to the network 102 for communication purposes. Such end user computer 106 may include a desktop computer, lap-top computer, and/or any other type of logic. Still yet, various other devices may be coupled to the network 102 including a personal mobile tablet 108, a mobile phone device 110, a television 112, etc.

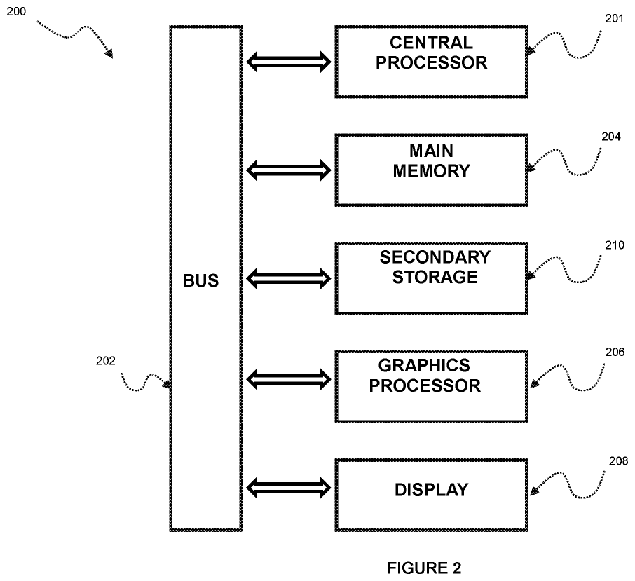

FIG. 2 illustrates an exemplary system 200, in accordance with one embodiment. As an option, the system 200 may be implemented in the context of any of the devices of the network architecture 100 of FIG. 1. Of course, the system 200 may be implemented in any desired environment.

As shown, a system 200 is provided including at least one central processor 201 which is connected to a communication bus 202. The system 200 also includes main memory 204 [e.g. random-access memory (RAM), etc.]. The system 200 also includes a graphics processor 206 and a display 208.

The system 200 may also include a secondary storage 210. The secondary storage 210 includes, for example, a hard disk drive and/or a removable storage drive, representing a floppy disk drive, a magnetic tape drive, a compact disk drive, etc. The removable storage drive reads from and/or writes to a removable storage unit in a well-known manner.

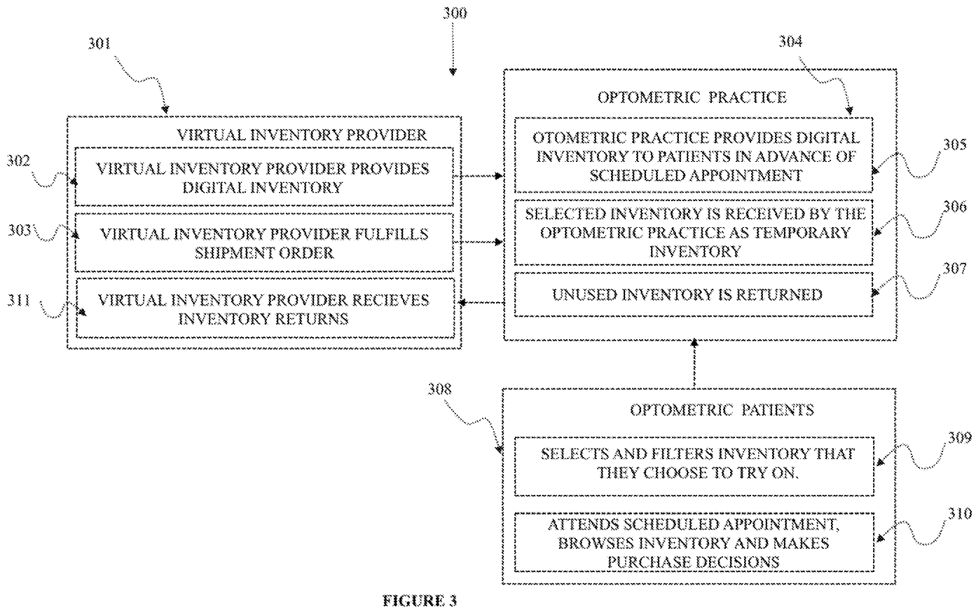

Computer programs, or computer control logic algorithms, may be stored in the main memory 204, the secondary storage 210, and/or any other memory, for that matter. Such computer programs, when executed, enable the system 200 to perform various functions (to be set forth below, for example). Memory 204, storage 210 and/or any other storage are possible examples of tangible computer-readable media. FIG. 3 illustrates a method 300 for enabling virtual inventory for optometric patients to review and present as available inventory in advance of their visit to the optometric practice. The patient selects eyewear favorites that they are interested in trying on while in the optometric practice. The selected inventory that does not reside in the physical inventory of the optometric practice is fulfilled and shipped to the practice.

As an option, the method 300 may be carried out in the context of the details of FIGS. 1 and/or 2. Of course, however, the method 300 may be carried out in any desired environment. Further, the aforementioned definitions may equally apply to the description below.

As shown, one or more virtual inventory providers 301 provides access to digital eyewear inventory for the optometric practice 302. At least one optometric practice 304 provides digital inventory including their physical inventory carried plus any number of virtual inventory as options in advance of the scheduled appointment 305. The optometric patient 308 reviews eyewear in advance and selects favorites 309. The virtual inventory provider 301 fulfills the patients selected frames for shipment to the optometric provider or any other address 303. The selected inventory is received at the optometric practice or other address as temporary inventory 306. The patient attends the scheduled appointment, browses inventory, Try's on frames including both normal physical inventory and temporary virtual inventory and makes purchasing decisions 310. Unpurchased virtual inventory can be returned to virtual inventory 307. The virtual inventory provider receives the returned virtual inventory 311.

1. A computer program product embodied on a non-transitory computer readable medium or mobile computing platform, comprising computer code for:
Enabling patients digital access to virtual inventories offered by vendors; to enable vendors such as optometric practices to carry virtual inventory in parallel with the optometry practices physical inventory offering; providing patient access to digitally delivered eyewear inventory who have a scheduled appointment with an optometric provider; digitally storing the current eyewear inventory found in an optometric practice including both physical inventory or virtual inventory; presenting the optometric practice's current eyewear inventory to patients on a mobile computing or internet-connected computing device; providing inventory options that are not currently in inventory; tracking consumer use of the virtual inventory software; fulfilling the inventory request so that the desired inventory will arrive at the optometric practice before the patients arrival.

2. The computer program product of claim 1, wherein the inventory provides alternatives that are not currently carried by the optometric practice.

3. The computer program product of claim 1, wherein the inventory is shipped to arrive in time for the patient's optometric appointment.

4. The computer program product of claim 1, wherein the physical inventory is optimized based upon the popular trends and sales so that the optometric practice is more likely to have the popular frames in their physical inventory.

5. The computer program product of claim 1, wherein virtual inventory is owned by either the optometric provider or by the virtual inventory provider.

6. The computer program product of claim 1, wherein the virtual inventory can be returned to the virtual inventory provider.

7. The computer program product of claim 1, wherein new inventory items can be offered for sale through virtual inventory and trialed to determine market viability and customer preference.

8. The computer program product of claim 1, wherein the digital inventory can be dynamically adjusted to only display inventory that can reasonably be fulfilled and shipped in time for the patients scheduled appointment. When there is not sufficient time to fulfill any shipments, the digital inventory may be limited to only show physical Inventory.

9. The computer program product of claim 1, wherein the virtual inventory bay be fulfilled and shipped to the optometric practice or any other address to reach the consumer.
